(19) United States
(12) Patent Application Publication
(10) Pub. No.: US 2012/0087983 A1
(43) Pub. Date: Apr. 12, 2012

(54) ORTHOPEDIC APPLICATION OF ENCAPSULATED STEM CELLS

(75) Inventors: Nathan Katz, Mount Prospect, IL (US); Felix Pustilnik, Walnut Creek, CA (US)

(73) Assignee: JOINTECHLABS, INC., Skokie, IL (US)

(21) Appl. No.: 13/245,639

(22) Filed: Sep. 26, 2011

Related U.S. Application Data
(63) Continuation-in-part of application No. 13/193,468, filed on Jul. 28, 2011.
(60) Provisional application No. 61/386,314, filed on Sep. 24, 2010.

(51) Int. Cl. A61K 9/14; A61P 19/02; A61P 19/04; A61K 35/12

(52) U.S. Cl. 424/489; 424/93.7

(57) ABSTRACT

Described herein are orthopedic applications of mesenchymal stem cell encapsulated and delivered for treatment of cartilage damage in joints. A therapeutic composition is prepared comprising a purified fraction of adipose-derived mesenchymal stem cells encapsulated in microbeads of a three-dimensional biocompatible gel matrix. The hydrogel microbeads encapsulating stem cells maintain the viability and location of the stem cells for an extended period as compared to stem cells in suspension. The microbeads are implanted adjacent a target orthopedic treatment site where the microbeads allow the release of cellular factors from the encapsulated stem cells to surrounding orthopedic tissues to achieve desired therapeutic results such as healing of cartilage damage in joints.

### CLAIM OF PRIORITY

This application is a continuation-in-part application of U.S. patent application Ser. No. 13/193,468 entitled “ENCAPSULATED ADIPOSE-DERIVED STEM CELLS, METHODS FOR PREPARATION AND THERAPUTIC USE” filed Jul. 28, 2011, and also claims priority to U.S. Provisional Patent Application 61/386,314, entitled “ORTHOPEDIC APPLICATION OF MESENCHYMAL STEM CELL ENCAPSULATION” filed Sep. 24, 2010; both of which are incorporated herein by reference.

### FIELD OF THE INVENTION

This invention relates generally to therapeutic compositions including stem cells derived from adipose tissue, including systems for preparing, delivering and utilizing such therapeutic compositions.

### BACKGROUND OF THE INVENTION

Regenerative medicine can be defined as harnessing the body's regenerative mechanisms in a clinically targeted manner, using them in ways that are not part of the normal healing mechanism or by artificially amplifying normal mechanisms. Stem cells are pluripotent or multipotent cells with the potential to differentiate into a variety of other cell types, which perform one or more specific functions and have the ability to self-renew. It has been found that stem cells from a variety of sources can be used for multiple therapeutic or prophylactic purposes. For example, hematopoetic stem cells (HSCs) derived from bone marrow are multipotent stem cells that can give rise to cell types from the myeloid (monocytes and macrophages, neutrophils, basophils, eosinophils, erythrocytes, megakaryocytes/platelets, dendritic cells), and lymphoid lineages (T-cells, B-cells, NK-cells). Mesenchymal stem cells (“MSCs”) derived from multiple tissues in the adult body are multipotent non-hematopoietic stem cells and are characterized by extensive proliferative ability in an uncommitted state while retaining the potential to give rise to cell types including osteoblasts, myocytes, chondrocytes, adipocytes, endothelial cells and beta pancreatic islet cells. MSCs are present in which arise from the embryonic mesoderm (e.g., hematopoietic cells and connective tissue). Thus, stem cells can be isolated from many tissue sources within the adult body.

Adipose tissue refers to fat including the connective tissue that stores the fat. Adipose tissue includes stem cells and endothelial precursor cells. As used herein, “adipose tissue” refers to a tissue containing multiple cell types including adipocytes and microvascular cells. It has been discovered that adipose tissue is an especially rich and practical source of mesenchymal stem cells. This finding is due, at least in part, to the ease of harvesting adipose tissue and the ease of removing the major non-stem cell component of adipose tissue, the adipocyte. In fact, a large quantity of mesenchymal stem cells can be obtained by simple aspiration from adipose tissue, for example, from lipoaspirate samples from aesthetic interventions. The lipoaspirate is typically centrifuged to separate the active cellular component from the mature adipocytes and connective tissue. The pellet containing the active cellular component (e.g., the component containing adipose-derived stem cells) is referred to as processed lipoaspirate (PLA).

Adipose-derived stem cells (ADSCs), methods for extracting such cells and methods for using such cells are disclosed for example in: Gimble et al., “Adipose-derived Stem Cells for Regenerative Medicine” Circ. Res. 100:1249-1260 (2007); Utsonomiya et al., “Human Adipose-Derived Stem Cells: Potential Clinical Applications in Surgery” Surg Today 41:18-23 (2011); Casteilla et al., “Adipose-derived stromal cells: Their identity and uses in clinical trials, an update” World J Stem Cells 3(4):25-33 (2011); U.S. Pat. No. 6,777,231 entitled “Adipose-Derived Stem Cells and Lattices” to Katz et al.; U.S. Pat. No. 7,901,672 entitled “Methods Of Making Enhanced Autologous Fat Grafts” to Fraser et al.; U.S. Patent Publication 2009/0304644 entitled “Systems And Methods For Manipulation Of Regenerative Cells Separated And Concentrated From Adipose Tissue” to Hedrick et al.; and U.S. Pat. No. 7,390,484 entitled “Self-Contained Adipose Derived Stem Cell Processing Unit” to Fraser et al., all of which are incorporated herein by reference.

### SUMMARY OF THE INVENTION

Mesenchymal stem cells (MSC) can differentiate into a variety of cell types including cells of connective tissues such as cartilage, muscle, adipose, or tendon. MSCs can be obtained from the bone marrow and can be expanded in vitro. Arthritis is a degenerative disease in which cartilage cells lose its function over time, leading to inflammation and other complications accompanied by the loss of cartilage surface on bones, ligaments and joints. Stem cells may be used for orthopedic application including treatment of treatment of cartilage damage in joints caused by osteoarthritis, aging, and/or mechanical injury.

The present invention relates generally to therapeutic compositions including stem cells, as well as systems for preparing, delivering and utilizing such therapeutic compositions in orthopedic applications. The stem cells may be administered to a patient as part of a therapeutic composition as described herein. In a preferred embodiment, the stem cells are provided in a three-dimensional platform. The three-dimensional platform includes adipose-derived stem cells encapsulated in a biocompatible hydrogel and formed into microbeads which protect and support the stem cells when introduced to the human body thereby enhancing therapeutic efficacy of the treatment as compared to stem cells in suspension. When implanted adjacent a target orthopedic treatment site the encapsulated stem cells provide extracellular therapeutic factors which migrate out of the hydrogel encapsulation into the surrounding tissues. The extracellular therapeutic factors promote rejuvenation of the surrounding tissues promoting reconstruction and healing of the target orthopedic treatment site by cells within and around the target treatment site. In particular the released extracellular factors can be used to promote healing of cartilage damage in joints caused by osteoarthritis, aging, and/or mechanical injury by rejuvenating tissues adjacent the damaged cartilage and causing them to repair the damage.

These and other objects, features and advantages of the invention will be apparent from the detailed description which follows.

### DETAILED DESCRIPTION OF THE PREFERRED EMBODIMENTS

Reference will now be made in detail to the presently preferred embodiments of the invention. Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. The present invention may be practiced in conjunction with various cell or tissue separation techniques that are conventionally used in the art, and only so much of the commonly practiced process steps are included herein as are necessary to provide an understanding of the present invention.

### Extraction and Isolation Of Stem Cells

Stem cells for utilization in the present inventions can be isolated from almost any embryonic or adult tissue including, but not limited to endothelial tissue from umbilical cord vein, endothelial tissue from foreskin, endometrial tissue, human embryonic stem cells, and adipose tissue. Pluripotent cells can also be artificially produced by inducing pluripotency such as described in Takahashi, K. & Yamanaka, S. Cell; 126: 663-676 (2006).

Mesenchymal Stem Cells (MSCs) are stem cells that can differentiate readily into lineages including osteoblasts, myocytes, chondrocytes, adipocytes, endothelial cells and beta pancreatic islet cells (Pittenger, et al., Science, Vol. 284, pg. 143 (1999); Haynesworth, et al., Bone, Vol. 13, pg. 69 (1992); Prockop, Science, Vol. 276, pg. 71 (1997)). MSCs, also known in the literature as bone marrow stem cells, skeletal stem cells, and multipotent mesenchymal stromal cells, are non-hematopoietic progenitor cells isolated from adult tissues, and are characterized in vitro by their extensive proliferative ability in an uncommitted state while retaining the potential to differentiate along various lineages of mesenchymal origin, including chondrocyte, osteoblast, and adipocyte lineages, in response to appropriate stimuli. In vitro studies have demonstrated the capability of MSCs to differentiate into muscle (Wakitani, et al., Muscle Nerve, Vol. 18, pg. 1417 (1995)), neuronal-like precursors (Woodbury, et al., J. Neurosci. Res., Vol. 69, pg. 908 (2002); Sanchez-Ramos, et al., Exp. Neurol., Vol. 171, pg. 109 (2001)), cardiomyocytes (Toma, et al., Circulation, Vol. 105, pg. 93 (2002); Fakuda, Artif. Organs, Vol. 25, pg. 187 (2001)) and possibly other cell types. MSCs are present in multiple tissues in the body which arise from the embryonic mesoderm (e.g., hematopoietic cells and connective tissue). As such, pluripotent cells useful for the present invention can be isolated from any of these tissue sources and can be induced to differentiate into any of these cell types.

In some embodiments, pluripotent cells are obtained from non-pathological post-natal mammalian adipose tissues. Pluripotent cells can be obtained from a source of adipose tissue, such as the stromal fraction of adipose tissue. The pluripotent cells can be obtained from any suitable source of adipose tissue from any suitable animal, including humans, having adipose tissue. For example, adipose tissue can be obtained by conventional techniques known for the skilled person in the art (e.g., liposuction), from any suitable source of adipose tissue from any suitable animal, including mammals such as dogs, cats, horses, pigs, cows and humans. Preferably, pluripotent stem cells utilized in the present invention are derived from a mammal, such as from a human. A convenient source of adipose tissue is from liposuction surgery. In fact, a large quantity of pluripotent cells can be obtained by simple aspiration from adipose tissue, for example, from lipoaspirate samples from aesthetic interventions. Because approximately 400,000 liposuction procedures are performed annually in the United States, this source of pluripotent cells, particularly “mesenchymal stem cells” (MSCs) is particularly promising for practicing the inventions disclosed herein.

In one instance, pluripotent cells of the invention are isolated from adipose tissue. A first step in any such method requires the isolation of the adipose tissue from the source animal. The animal can be alive or dead, so long as adipose stromal cells within the animal are viable. Typically, human adipose tissue is obtained from a living donor, using well-recognized protocols such as surgical or suction lipectomy. The preferred method to obtain human adipose tissue is by excision or liposuction procedures well known in the art. The pluripotent cells of the invention are present in the initially excised or extracted adipose tissue, regardless of the method by which the adipose tissue is obtained.

From whatever source, the tissue source containing pluripotent cells is processed to separate the pluripotent cells of the invention from the remainder of the tissue. Pluripotent cells can be obtained by washing the tissue with a physiologically-compatible solution, such as phosphate buffer saline (PBS). Typically, a washing step consists of rinsing the adipose tissue with PBS, agitating the tissue, and allowing the tissue to settle. In addition to washing, the adipose tissue can be dissociated. Dissociation can occur by enzyme degradation (e.g., trypsin treatment). Alternatively, or in conjunction with such enzymatic treatment, other dissociation methods can be used such as mechanical agitation, sonic energy, or thermal energy. Cells are then centrifuged and the pellet (containing the pluripotent cells) is further treated after resuspension in an appropriate solution (e.g., PBS).

Pluripotent cells in the resuspended pellet can be separated from other cells of the resuspended pellet by methods that include, but are not limited to, cell sorting, size fractionation, granularity, density, molecularly, morphologically, and immunohistologically (e.g., by panning, using magnetic beads, FACS, MACS, or affinity chromatography. In some immunologically-based methods of cell isolation, a pluripotent cell is obtained by positive selection, via the use of an antibody or other specific-binding protein, which binds to an epitope on the cell surface. The step of recovering the cells from the antibodies is performed by washes with suitable buffers, known to one skilled in the art. Alternately, pluripotent cells can be isolated by negative selection. The presence of pluripotent cells may be verified and assessed using specific cell surface markers which are identified with unique monoclonal antibodies, for example, see U.S. Pat. No. 5,486,359.

Pluripotent cells of the invention are also capable of being expanded in vitro. That is, after isolation, said cells can be maintained and allowed to proliferate in culture medium. Such medium is composed of any suitable cell medium, for example, Dulbecco's Modified Eagle's Medium (DMEM), with or without antibiotics, and glutamine, and/or supplemented with fetal bovine serum (FBS). It is within the skill of one in the art to modify or modulate concentrations of media and/or media supplements as necessary for the cells used. The maintenance conditions of pluripotent cell population(s) of the invention can also contain cellular factors that allow cells to remain in an undifferentiated form. In vitro expansion of pluripotent cells without inducing differentiation can be accomplished for example by using specially screened lots of suitable serum (such as fetal bovine serum or human serum). Any of the steps and procedures for isolating pluripotent cells of the invention can be performed manually, for example by microscopic evaluation based on phenotype and/or marker expression. Alternatively, the process of isolating such cells can be facilitated and/or automated through one or more methods known in the art, for example, FACS or MACS sorting. Assessment of the stem cell population prior to incorporation into a pharmaceutical composition advantageously allows quantification and characterization of the extracted stem cell population in order to control the number of stem cells incorporated into a therapeutic composition thereby facilitating delivery of a desired prophylactically or therapeutically effective amount of stem cells to the patient.

Particular methods for extracting and processing adipose tissue are disclosed in Applicants' copending U.S. Patent Application U.S. patent application Ser. No. 13/193,468 entitled “ENCAPSULATED ADIPOSE-DERIVED STEM CELLS, METHODS FOR PREPARATION AND THERAPUTIC USE” filed Jul. 28, 2011 incorporated herein by reference.

### Therapeutic Compositions Of Stem Cells

For the administration in the prevention and/or treatment of a disorder, such as joint disorders (e.g., osteoarthritis), cells of the invention can be formulated in a suitable pharmaceutical composition, comprising cells of the invention, in a therapeutically or prophylactically effective amount, together with a suitable pharmaceutically acceptable vehicle. The pharmaceutical composition of the invention can be formulated according to the chosen form of administration. For example, a pharmaceutical composition is prepared in a liquid dosage form, e.g., as a suspension, to be injected into the subject in need of treatment. The pharmaceutical composition of the invention can contain a prophylactically or therapeutically effective amount of the cells of the invention, preferably in a substantially purified form, together with the suitable vehicle in the appropriate amount in order to provide the form for proper administration to the subject. In a preferred embodiment, a therapeutic composition includes stem cells encapsulated in hydrogel microbeads as part of a 3-dimensional platform as described below and suitable for subchondral introduction adjacent a target orthopedic treatment site.

As used herein the term “prophylactically or therapeutically effective amount” refers to the amount of cells of the invention contained in the pharmaceutical composition which is capable of producing the desired therapeutic effect. One of skill in the art will recognize that cell numbers (e.g., dosage amount) will vary depending upon multiple factors including, but not limited to site of administration, extent of disease, and method of administration. For example, an administration directly into the joint of a subject suffering from OA will advantageously require a smaller number of cells than an administration of the cells into the bloodstream. The dose of cells disclosed herein can be repeated, depending on the patient's condition and reaction, at time intervals of days, weeks or months as determined necessary by a treating physician or other healthcare professional. As previously described, assessment of the extracted stem cell population prior to incorporation into a pharmaceutical composition allows quantification and characterization of the extracted stem cell population in order to control the number of stem cells incorporated into a therapeutic composition thereby facilitating delivery of a desired prophylactically or therapeutically effective amount of stem cells to the patient.

The term “pharmaceutically acceptable vehicle” refers to a composition approved by a regulatory agency of the Federal or a state government or listed in the U.S. Pharmacopeia, or European Pharmacopeia, or other generally recognized pharmacopeia for use in animals, including humans. The term “vehicle” refers to a diluent, adjuvant, excipient, or carrier with which the cells of the invention are administered, thus, the vehicle must be compatible with the cells. Examples of suitable pharmaceutical vehicles are described in “Remington's Pharmaceutical Sciences” by E. W. Martin. Illustrative, non-limiting, examples of vehicles for the administration of cells contained in a pharmaceutical composition of the invention include, for example, a sterile saline solution (0.9% NaCl), PBS, etc.

The pharmaceutical compositions of the invention, if desired, can also contain, when necessary, additives to enhance, control, or otherwise direct the intended therapeutic effect of the cells comprising said pharmaceutical composition, and/or auxiliary substances or pharmaceutically acceptable substances, such as minor amounts of pH buffering agents, tensioactives, co-solvents, preservatives, etc. For example, the pharmaceutical composition preferably comprises constituents which protect, culture, and maintain the stem cells for a desired treatment period of 5 to 14 days or more thereby extending the release of therapeutic extracellular factors from the encapsulated cells. The pharmaceutical composition can also contain constituents to maintain the stem cells in undifferentiated form. The stability of the cells in the pharmaceutical composition of the invention can be improved by means of adding additional substances, such as, for example, amino acids such as aspartic acid, glutamic acid, etc. Pharmaceutically acceptable substances that can be used in the pharmaceutical composition of the invention are known, in general, by the skilled person in the art and are normally used in the manufacture of cellular compositions.

In one example of a therapeutic composition, chondrocyte-like cells are produced from isolated pluripotent cells by any of the methods described herein. Chondrocyte-like cells are then prepared for application to subjects in need of the cells. Chondrocyte-like cells can also be prepared in pharmaceutical dosages (e.g., in a pharmaceutically acceptable solution) and stored in appropriate containers. The chondrocyte-like cells can be stored in an appropriate manner (e.g., frozen) until needed. Additionally, the pharmaceutical dosages can be placed in pre-prepared syringes, catheters or other medical devices appropriate for delivery to an affected joint. One of skill in the art will recognize that dosage amount, needle length and other such parameters can be adjusted for any individual preparation.

A pharmaceutical composition containing cells of the present invention may be stored until use by means of conventional methods known by the skilled person in the art. For short term storage (less than 6 hours) the pharmaceutical composition containing said cells may be stored at or below room temperature in a sealed container with or without supplementation with a nutrient solution. Medium term storage (less than 48 hours) is preferably performed at 2-8° C., the pharmaceutical composition comprising an iso-osmotic, buffered solution in a container composed of or coated with a material that prevents cell adhesion. Longer term storage is preferably performed by appropriate cryopreservation and storage under conditions that promote retention of cellular function.

Cells in either prepared dosages or pre-dosage containers can be shipped to medical facilities through any approved delivery system (governmentally approved and/or commercial). Cells can be delivered directly from the manufacturer or via an intermediary. Fees can be collected for delivery of the cells through any appropriate means (e.g., credit card, credit account, cash, check, etc.).

### Encapsulation Of Stem Cells—3D Platform

In an embodiment of the invention, a 3-dimensional (3D) platform is created to support cells introduced as a therapeutic composition into the body. In general terms the platform is created by extraction of SVM with or without purification of stem cells. The cells are dispersed in a biocompatible gel/polymeric matrix. Preferably the gel is biocompatible and biodegradable, for example a fibrin hydrogel. T gel composition preferably comprises constituents which protect, culture, and maintain the stem cells for a desired treatment period of 5 to 14 days or more thereby extending the release of therapeutic extracellular factors from the encapsulated cells. The pharmaceutical composition can also contain constituents to maintain the stem cells in undifferentiated form.

The gel containing the stem cells is preferably formed into microbeads thereby increasing the active surface area of the therapeutic composition. The formations of microbeads promotes the release of extracellular factors produced by the encapsulated stem cells into the surrounding tissues. The formation of the microbeads can be performed manually or mechanically. Preferably, the microbeads are of 10 to 50 μL in size and each bead includes from 2,000 to 10,000 stem cells. The concentration of stem cells in the microbeads is selected so as to be high enough to achieve the desired therapeutic effect without reduced the effectiveness of the encapsulation and viability of the stem cells. A suitable concentration of stem cells has been found to be 200 stem cells per μL. This concentration of stem cells has been found to promote maintenance and viability of the stem cells within the microbeads for up to 14 days. The therapeutic composition of microbeads of gel containing stem cells can then be injected or applied superficially as a therapeutic composition or to augment autologous adipose tissue transplant procedures. In a preferred embodiment the microbeads are injected into subchondral bone adjacent to a target orthopedic site, such as a lesion in cartilage, in order to promote healing/growth of cartilage.

In one embodiment, an automated process is used for the creation of microbeads. The process includes utilization of disposable chip-device comprising two micro channels, a pump and a droplet formation system. Pumping mix of cells with components of gel matrix (fibrinogen, for instance) through one channel, while polymerization solution (thrombin, for instance) through another, allows creation of encapsulated cells platform in polymeric encapsulation matrix (fibrin in our example). The components combine into a mixture and the mixture is then broken into micro-droplets of particular size with a precise concentration of cells per droplet. The concentration of stem cells in the microbeads is selected so as to be high enough to achieve the desired therapeutic effect without reduced the effectiveness of the encapsulation and viability of the stem cells. A suitable concentration of stem cells has been found to be 200 stem cells per μL. The volume of the droplets can be controlled by adjusting the pumping rate and the frequency of the droplet formation system. Preferably, the microbeads are of 2 to 50 μL in size and each bead includes from 400 to 10,000 stem cells. In one embodiment, the droplet formation system is operated at ˜5 Hz (20 μL/sec) and produced droplets/microbead of 4 μL in volume containing about 1,600 stem cells. The system can readily be operated at 50 Hz (200 μL/sec) and above.

The 3D platform provides significant advantages compared to a dispersed suspension of stem cells. For example, the stem cells remain in groups, keeping on interaction, normal proliferation and gross factor secretion whereas in suspension, the single stem cells are unable to sustain normal development in form of single cells in suspension. Additionally, the 3D platform defends the encapsulated stem cells against environmental changes and mechanical stress upon delivery of the stem cells into the hosting tissue. Where allogeneic stem cells are used, the 3D matrix prevents and/or delays development of an immune response to the cells promoting their viability during the treatment period. The 3D matrix supports the stem cells assuring normal metabolism over an extended period as compared to a suspension of stem cells.

The support of the stem cells by the 3D platform also enhances the storage or cryostorage of the stem cells facilitating maintenance, transport and delivery of stem cells at the time and place required for a procedure. Additionally, the initial amount of available and injected cells can be determined during preparation of the 3D platform, which allows development of dose-dependent controlled treatment. This is facilitated, as described above, by characterization of the stem cell population prior to and/or during preparation of the therapeutic composition.

The advantages of the 3D platform provide significant therapeutic benefits. The encapsulated stem cells in the 3D platform can be more accurately delivered to a precise location in the human body and will remain in the targeted location—as opposed to stem cells in suspension which rapidly dissipate from the injection site. Moreover, as described above, the 3D platform increases the longevity and functionality of the stem cells by protecting them against chemical, mechanical and immunogenic stress at the injection site. Maintaining the stem cells in the target location and extending their viability extends the treatment effects of the stem cells thereby enhancing the treatment and or reducing the need for repetition of the treatment.

In a preferred embodiment adipose-derived stem cells are encapsulated in the 3D platform to generate a therapeutic composition. Preparation of encapsulated stem cells requires three general steps. First, stem cells must be extracted, and isolated. The extraction and isolation of stem cells can be performed using conventional methods and/or the methods described herein. Second, the stem cells are mixed with a liquid phase biocompatible pro-polymer. Third, the mixture is caused to gel by crosslinking of the pro-polymer to form a polymer. As result, the stem cells are embedded in polymeric biodegradable hydrogel network which serves as 3D culture and support system for the stem cells. A range of biocompatible polymers are know to those of skill in the art including, for example, fibrin, alginate and collagen polymers. A suitable polymer is biocompatible and biodegradable but provides suitable mechanical and chemical support to the stem cells during a period over which they can have therapeutic effect. In a preferred embodiment, a Fibrin/thrombin gel is used suitable for maintaining the stem cells for a period of three to fourteen days.

A therapeutic composition comprising encapsulated stem cells can be introduced into tissues adjacent the site of an injury. For example the composition can be introduced into subchondral bone adjacent the site of a cartilage injury. Because the cells are encapsulated they do not migrate away from the site of introduction. However, the stem cells can persist and/or proliferate in vivo within the matrix. Moreover, the presence of the encapsulated stem cells can stimulate growth/proliferation of tissues adjacent the encapsulated stem cells by, for example, the release of cytokines, growth factors and anti-inflammatory factors. It is thought that encapsulated stem cells introduced in this manner can achieve regenerative healing without differentiation and integration of the stem cells actually introduced. Indeed, in some embodiments, encapsulation of the stem cells extends the period of the treatment effect by maintaining the stem cells in undifferentiated form isolated from factors in the tissue which might engender differentiation of the stem cells. Thus, in some embodiments it is desirable to introduce the stem cells adjacent the site of an injury rather than directly at the site of an injury. In a preferred embodiment, for example, encapsulated stem cells are introduced into subchondral bone where the factors released by the encapsulated stem cells migrate into the surrounding subchondral tissues stimulating production of new cartilage tissue.

### Administration Of Therapeutic Compositions

The administration of the pharmaceutical composition of the invention to the subject in need thereof can be carried out by conventional means. In a particular embodiment, said pharmaceutical composition can be administered to the subject in need by administration using devices such as syringes, catheters, trocars, cannulae, etc. In any case, the pharmaceutical composition of the invention will be administrated using the appropriate equipments, apparatus, and devices which are known by the skilled person in art in a therapeutically or prophylactically effective amount, together with a suitable pharmaceutically acceptable vehicle.

Cells disclosed herein can be applied by several routes including direct injection into the affected anatomical site. A pharmaceutical composition containing the cells may be injected in a single bolus, through a slow infusion, or through a staggered series of applications separated by several hours, several days or weeks. In any case, the pharmaceutical composition of the invention will be administrated to the target tissue using the appropriate equipments, apparatus, and devices which are known by the skilled person in art in a therapeutically or prophylactically effective amount.

One of skill in the art will recognize that cell numbers (e.g., dosage amount) will vary depending upon multiple factors including, but not limited to site of administration, extent of disease, and method of administration. For example, an administration directly into the joint of a subject suffering from OA will typically contain a smaller number of cells than an administration of the cells into the bloodstream. The dose of cells disclosed herein can be repeated, depending on the patient's condition and reaction, at time intervals of days, weeks or months as determined necessary by a treating physician or other healthcare professional.

In a preferred embodiment, stem cells are encapsulated as part of a 3D platform as described above. The 3D platform incorporating the stem cells are then delivered to a target location to achieve the desired therapeutic effect. The 3D platform can be administered to the subject in need by direct administration into target tissue using devices such as syringes, catheters, trocars, cannulae, etc. In a preferred embodiment the 3D platform is administered percutaneously under image guidance to a desired location.

One mode of treatment introduces stem cells encapsulated in the 3D platform adjacent tissues to be treated. The 3D platform maintains the stem cells in their undifferentiated from and protects the stem cells from chemical and mechanical stress at the site of introduction. The encapsulated stem cells are able to survive and/or proliferate in vivo for an extended period as compared to stem cells in suspension. The encapsulated stem cells release cytokines, growth factors, anti-inflammatory factors which migrate out of the 3D platform into the surrounding tissues. These factors engender a therapeutic effect in the target tissues adjacent the site of injection of the 3D platform.

In one example, a 3D platform encapsulating stem cells is used to treat osteoarthritis. Rather than injecting a suspension of stem cells directly into a joint, the 3D platform is introduced sub-chondrally using an image-guided needle or similar technology. The stem cells in the 3D platform do not themselves differentiate into tissues to repair the joint. However cellular factors released from the 3D platform are able to migrate into the osteo-arthritic tissues of the joint rejuvenating the tissues and thereby stimulate those tissues to repair themselves. Essentially the 3D platform permits the release of factors from the encapsulated stem cells with reduce inflammation and trigger self repair in the affected tissues of the joint such as the damaged cartilage. Triggering self-repair of the tissues allows for creation of defect repairs which more closely approximate the natural tissue. This is advantageous to direct injection of stem cells to differentiate into repair tissue because such approach generally results in tissues that doe not have the desired structural features of the natural tissues.

Cells and pharmaceutical compositions of the present disclosure can be used in a combination therapy with other substances useful for treating the same disorder. Such combination therapy can comprise the cells of the present invention directly combined with other substances (e.g., other pharmaceuticals), or in conjunction with other substances. Combination therapy can also include delivery of therapeutic compositions as described herein along with

The cells disclosed herein can be cells of autologous or allogeneic origin. Autologous stem cells are derived from the individual into whom the stem cells are later reinfused. Autologous stem cells are advantageous in that, being the individuals own tissue, they do not engender an immune response from the recipient. Allogeneic stem cells on the other hand, are derived from one or more individuals other than the recipient. These stem cells can illicit an immune/rejection response in some circumstances. Steps can be taken to reduce the chance of rejection, such as by using tissue-matched donors. Allogeneic stem cells can still be utilized in certain applications. Although autologous stem cells are preferred, allogeneic stem cells can, for example be utilized as part of a 3D-platform described above—the allogeneic stem cells are effective to deliver therapeutic cellular factors for a period after introduction to the patient and are protected from rejection by the 3D platform at least for the limited period during which they have their therapeutic effect.

### Orthopedic Application Of Encapsulated Stem-Cells

Mesenchymal stem cells (MSC) can differentiate into a variety of cell types including cells of connective tissues such as cartilage, muscle, adipose, or tendon. MSCs can be obtained from the bone marrow and can be expanded in vitro. Arthritis is a degenerative disease in which cartilage cells lose its function over time, leading to inflammation and other complications accompanied by the loss of cartilage surface on bones, ligaments and joints.

Described herein is an application of encapsulated mesenchymal stem cells (MSCs) for treatment of cartilage damage in joints. In a preferred embodiment, for example, encapsulated stem cells are introduced into subchondral bone where the factors released by the encapsulated stem cells migrate into the surrounding subchondral tissues stimulating production of new cartilage tissue. Cell encapsulation/inoculation is the introduction of cells into a semi-permeable membrane that allows diffusion of small molecules. There are several beneficial effects of cell encapsulation/inoculation. First, it protects the cell from mechanical stress, which is important in case of cartilage restoration in the joint. Second, it forms three-dimensional culture system, which, as it has been reported, tremendously speeds up the process of cell differentiation. Even though mesenchymal stem cells possess previously described unique biological feature as capability of depressing immune reaction, encapsulation can serve as additional barrier between cells and hosting immune system. That can be important in allogeneic clinical applications of mesenchymal stem cells.

There are many polymeric biodegradable biomaterials that are used for encapsulation. Encapsulation is performed by the following steps: predetermined and/or undifferentiated MSC will be embedded into gel-like viscous substance, which is introduced into joint by orthopedic technique(s). This substance would be applied to the targeted spot of cartilage damage.

Additional bio and mechanical techniques would be applied in order to allow safe development of the cells in situ. For example, bio film covering the site or other orthopedic methods allowing reduction of the pressure on the healing spot of cartilage can be used.

Additional information regarding isolation, purification and therapeutic benefits of stems cells can be found in the following references which are incorporated herein by reference. Incorporated herein are the following references to the same extent as if each individual publication, patent, or patent application was specifically and individually indicated: U.S. Pat. Nos. 6,153,432; 6,214,369; 6,387,367; 6,429,013; 6,555,374; 6,777,231; 6,835,377; 6,841,150; 7,470,537; U.S. Pat. Application Nos. 2008/0112936, 2008/0260694, 2008/0317718, 2009/0148419, 2009/0162934; and Murphy et al., Arthritis & Rheumatism, 48: 3564-3474 (2003); Lee et al, Stem Cells, 25: 2964-2971 (2007); Ishimura et al., Tohoku J. Exp. Med., 216:149-156 (2008); Hashemibeni et al., Iranian J. Basic Med. Sci., 11:10-17 (2008); Gimble et al., Circulation Research, 100: 1249-1260 (2007); Chen et al., Arthritis Research & Therapy 1 or 223 (2008); Astori et al., J. Translational Med., 5:55 (2007); Angioblast Revascor Press Release, “Proprietary technology in world-first allogeneic stem cell trial for treatment of end-stage heart failure in patients supported by mechanical heart assist devices”, August 5, 2009 (available at http://www.angioblast.com); Aggarwal et al., Blood, 105: 18 15-1822 (2005).

In on embodiment adipose-derived stem cells are encapsulated in a three-dimensional gel to form a 3D platform prior to introduction into a patient. Treatment with encapsulated stem cells provides significant biologic advantage over treatment with a dispersed suspension of stem cells. For example, the stem cells remain in groups, keeping on interaction, normal proliferation and gross factor secretion whereas in suspension, the single stem cells are unable to sustain normal development in form of single cells in suspension. Additionally, the matrix of the 3D platform defends the encapsulated stem cells against environmental changes and mechanical stress upon delivery of the stem cells into the hosting tissue. The matrix of the 3D platform supports the stem cells assuring normal metabolism. Advantageously, in the present example, the initial amount of available and injected cells is known to patient and physician, which allows development of dose-dependent controlled treatment.

Preparation of encapsulated stem cells requires three general steps. First, stem cells must be extracted, and isolated. The extraction and isolation of stem cells can be performed using conventional methods and/or the methods described herein. Second, the stem cells are mixed with a liquid phase biocompatible pro-polymer. Third, the mixture is caused to gel by crosslinking of the pro-polymer to form a polymer. As result, the stem cells are embedded in polymeric biodegradable hydrogel network which serves as 3D culture and support system for the stem cells.

In one embodiment, stem cells are derived from adipose tissue as follows. Adipose tissue is collected from a patient by mini liposuction into a sterile syringe in amount of 50-100 ml in a doctor's office. The extracted adipose tissue in the sterile syringes is processed on site or transported to a nearby facility for processing. The contents of the sterile syringes is then released into sterile 50 ml tubes and spun in a centrifuge at 200 g for 5 min. After centrifugation, the fraction of white fat is removed. The cell fraction is weighed and Liberase (Roche) is added in final concentration 12 mg/ml (0.28 Wunsch/ml). The cell fraction is digested with Liberase for 30 minutes in 36.6° C. in hot air shaker. At the end of 30 minutes the digestion is halted by addition of DMEM medium supplemented 15% human plasmanate (commercial). The treated cell fraction is the washed twice with reconstituted “StemPro”—a non-animal source recombinant medium (Invitrogen). The pellet containing the cell fraction is then plated in 75 cm² flask and incubated overnight in 5.5% CO2 incubator at 36.6° C. After overnight incubation cells are removed by exposition to “TrypLE Select”-recombinant trypsin (Invitrogen) for 8 min followed by washing in reconstituted StemPro medium. After washing, the stem cells are resuspended in 1 ml of StemPro medium. The suspension of stem cells can be characterized at this point by counting the cells using, e.g., a Scepter automated cell counter. A 100 μL sample of the suspension is removed for flow cytometry cell characterization and microbiology testing.

The remaining 900 μL of the stem cell suspension is used for encapsulation as follows. A 5 ml solution of Fibrinogen is prepared in concentration 10 mg/ml in tris-based DPBS.2. Stem cells in suspension are added to the solution of Fibrinogen to achieve final concentration of 200,000 cells/ml by gentle pipetting. The amount of stem cell solution to be added can be calculated because the stem cell solution has previously been characterized/counted. The mixture of the fibrinogen precursor and stem cells is the pipetted into 10 ml of thrombin solution in concentration 50 mg/ml. The resulting mixture is incubated in 5.5% CO2 at 36.6° C. for thirty minutes to form a gel including the encapsulated stem cells.

In a preferred embodiment the gel is formed into microbeads by manual or using an automatic chip device described above. The microbeads are preferably 10-50 μL in size. The microbeads are designed and the stem cell concentrated selected such the microbeads can support and maintain the stem cells in vivo for a period of 3 to 14 days during which period the stem cells remain within the microbead and release therapeutic factors into the surrounding tissues. The microbeads are kept in incubator until 2 hrs before scheduled procedure. At that point the product is delivered to Surgical Center at ambient temperature. Alternatively, the encapsulated stem cells are frozen and delivered in ready-to-use condition. Microbeads can be frozen by slow freezing or vitrification method and delivered in ready-to-use condition. Alternatively, the stem cells can be extracted and the 3-D platform and microbeads can be created at the site of the procedure using appropriate devices.

Treatment of osteoarthritis is performed by injecting encapsulated stem cells in the form of the 3D platform. The 3D platform is created as described above utilizing a fibrin gel encapsulating adipose-derived autologous stem cells. (Note that in alternative embodiments stem cells of other types-and origin can be utilized). The 3D platform microbeads incorporating the stem cells are then injected sub-chondrally adjacent damaged tissues such as damaged cartilage of an affected knee or elbow joint. Additional bio and mechanical techniques would be applied in order to allow safe development of the cells in situ. For example, bio film covering the site or other orthopedic methods allowing reduction of the pressure on the healing spot of cartilage can be used.

The administration of the 3D platform can be carried out by conventional means. In a particular embodiment, the 3D platform is introduced sub-chondrally using devices such as syringes, catheters, trocars, cannulae, etc. The administration can be performed percutaneously/arthroscopically and can be repeated as necessary to achieve the desired therapeutic effects. For example, the 3D platform is introduced sub-chondrally using an image-guided needle or similar technology. In any case, the pharmaceutical composition of the invention will be administrated using the appropriate equipments, apparatus, and devices which are known by the skilled person in art in a therapeutically or prophylactically effective amount.

The microbeads maintain the stem cells in the target location in undifferentiated from and protect the stem cells from chemical and mechanical stress at the site of introduction. The encapsulated stem cells are able to survive and/or proliferate in vivo within the microbeads for an extended period as compared to stem cells in suspension introduced into the joint. In a preferred embodiment the encapsulated stem cells are maintained for 3-14 days. The encapsulated stem cells release cytokines, growth factors, anti-inflammatory factors which migrate out of the 3D platform into the surrounding tissues. These factors engender a therapeutic effect in the target tissues adjacent the site of injection of the 3D platform. The cellular factors released from the 3D platform are able to migrate into the osteo-arthritic tissues of the joint rejuvenating the damaged tissues and thereby stimulating those tissues to repair themselves and achieve a therapeutic result. At the end of the 3-14 days the fibrin of the microbeads is digested and the stem cells are no longer maintained. The procedure can be repeated as necessary to achieve the desired therapeutic effect.

Any feature or combination of features described herein are included within the scope of the present invention provided that the features included in any such combination are not mutually inconsistent as will be apparent from the context, this specification, and the knowledge of one of ordinary skill in the art. For purposes of summarizing the present invention, certain aspects, advantages and novel features of the present invention have been described herein. Of course, it is to be understood that not necessarily all such aspects, advantages or features will be embodied in any particular embodiment of the present invention. Additional advantages and aspects of the present invention are apparent in the following detailed description and claims.

A number of publications and patents have been cited hereinabove. Each of the cited publications and patents are hereby incorporated by reference in their entireties. All publications, patents and patent applications are herein incorporated by reference in their entirety to the same extent as if each individual publication, patent or patent application was specifically and individually indicated to be incorporated by reference in its entirety.

The foregoing description of preferred embodiments of the present invention has been provided for the purposes of illustration and description. It is not intended to be exhaustive or to limit the invention to the precise forms disclosed. Many embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the claims and their equivalents.

What is claimed is:

1. A method for treating a patient comprising:
(a) receiving a therapeutic composition which includes a plurality of microbeads of a biocompatible hydrogen wherein each microbead contains a plurality of stem cells encapsulated within the hydrogel;
(b) introducing said therapeutic composition in subchondral bone adjacent a defect in cartilage; and
(c) causing the release of therapeutic cellular factors from said stem cells encapsulated within the hydrogel to said subchondral bone to promote regeneration of cartilage to fill said defect.

2. The method of claim 1, wherein the defect in cartilage is the result of osteoarthritis.

3. The method of claim 1, wherein the defect in cartilage is the result of aging.

4. The method of claim 1, wherein the defect in cartilage is the result of trauma.
